(19) United States
(12) Patent Application Publication
(10) Pub. No.: US 2012/0039901 A1
(43) Pub. Date: Feb. 16, 2012

(54) METHOD FOR THE PREVENTION AND TREATMENT OF CANCER BY INHIBITION OF GPVI

(75) Inventors: Ulrich Kronthaler, Deisenhofen (DE); Bernhard Nieswandt, Eibelstadt (DE)

(73) Assignee: CSL BEHRING GmbH

(21) Appl. No.: 13/247,645

(22) Filed: Sep. 28, 2011

Related U.S. Application Data
(62) Division of application No. 12/988,956, filed on Oct. 21, 2010.

(30) Foreign Application Priority Data
Apr. 22, 2008 (EP) 08007754.8

(51) Int. Cl. A61K 39/395; A61P 35/00; G01N 33/50

(52) U.S. Cl. 424/143.1; 424/174.1; 436/63

(57) ABSTRACT

The invention provides an inhibitor of GPVI for the prevention and or therapy of cancer and also the use of such an inhibitor of GPVI in the manufacture of a medicament for the prevention and/or therapy of cancer. A further aspect of the invention are pharmaceutical formulations comprising a GPVI inhibitor, which are suitable for the treatment of cancer, preferably for skin cancer, more preferably for melanoma, and most preferably for malignant cutaneous melanoma.

Cancer represents still one of the key challenges for medicine. Over the recent years, substantial research has resulted in options of specific therapies for specific types of cancer. Examples for specific malignancies to be treated with specific therapies are breast cancer to be treated by Herceptin®, and for other variants, such as progressed rectal or lung cancer to be treated with Avastin®, progressed rectal cancer or head and neck cancer with Erbitux®, chronic lymphocytic leukaemia with MabCampath® or follicular non-Hodgkin lymphoma to be treated with Zevalin®. Yet, none of these specific therapies was capable to fully replace classical chemotherapy or surgical interventions. In fact most of the new therapies are insufficiently effective on their own, thus requiring chemotherapy as co-medication. Chemotherapy on the other hand inevitable brings the burden of severe and possibly serious side effects. In brief, treatment options for cancer are still limited and are, if available at all, substantially less tolerable as compared to the standard therapy of other life threatening diseases such as cardiovascular diseases.

While the choice of therapeutic options to treat cancer is limited and associated with adverse side effects, for some very aggressive malignancies, the disease progress is so rapid, that the diagnosis frequently comes too late to expect a reasonable benefit even from an immediate start of classical therapy.

One of these aggressive variants is malignant cutaneous melanoma, a skin cancer. Several risk factors have been identified for development of such melanoma. Besides genetic predisposition, skin-tone and hair-colour play a role. Most frequently affected are light-skinned persons, i.e. with a light-sensitive type of skin. A special risk confers the gene for a reddish hair-colour. In comparison to the risk of people with these predispositions, the risk of dark-skinned persons to develop skin cancer is just 10%. Not only the progress of this type of cancer is rapid, also the number of patients affected is substantial. Each year there are about 15,000 new cases just in Germany, and about 2,000 patients die from the consequences. This is significantly more than all fatalities caused by other skin tumors and currently the mortality rate from melanomas rapidly increases above those of any other cancer. Diagnosed at a very early stage, the chances for a long-term cure by a complete surgical removal of the primary tumor are still high. In contrast to other malignancies, melanoma has, however, at the point in time when the disease is diagnosed, typically already widely spread in the body by metastasis. Surgical removal is then not possible any more, leaving radiation- or systemic chemotherapy as the only yet insufficiently effective therapeutic strategies so far. This high invasiveness and metastasis makes malignant melanoma extremely virulent, both in comparison to other forms of skin cancer as well as in comparison to other variants of cancer in general. Metastasis at a progressed state with other malignancies is similarly devastating for the future course of the disease. Accordingly, prognosis is worse in those patients in which circulating melanoma or other cancer cells were detected. Once multiple organs are affected, the chances for success by current therapeutic interventions substantially decrease. Irrespective of the type of cancer and of the point in time when metastasis occurs with specific tumors, the malignant cells typically spread via the vascular system.

Platelets play a key role in the processes of hemostasis and thrombosis. They arrest and become activated at sites of lesion due to their interaction with subendothelial collagen, which gets exposed by the lesion. The interaction between collagen and platelets is essentially mediated by three receptors: 1) alpha2beta1 integrin receptor which primary role is adhesion of platelets to collagen 2) GPIb-V-IX which binds to collagen receptor indirectly via Von Willebrand Factor (VWF) and 3) GPVI, a signalling receptor, activating platelets upon binding to collagen. GPVI is a member of the immunoreceptor family and co-expressed on platelets with Fc receptor gamma-chain (FcRgamma). Ligand-binding to GPVI leads to the activation of platelets and thereby mediates platelet aggregation (Varga-Szabo D. et al., Arterioscier. Thromb. Vasc. Biol. 2008, Vol. 28(3), 403-412). Taken together, the alpha2beta1 and GPIb-V-IX receptor as well as GPVI were primarily regarded as a promising target for the prevention arterial thrombosis resulting from cardiovascular or other diseases (Clemetson K. J. et al., 2007, Curr. Pharm. Des., vol. 13(26), 2673-2683)). GPVI was the most promising therapeutic target because a single treatment can cause a long term loss or inhibition of GPVI (Nieswandt B. et al., J. Exp. Med. 2001, Vol. 193(4): 459-469).

With the interaction of platelets and collagen playing a key role in the processes of hemostasis and thrombosis, it was not too surprising, that the inhibition of these three collagen receptors results in anti-thrombotic effects. In addition to the function of platelets for thrombosis and hemostasis, they were recently identified as important players in cancer growth and metastasis. Due to the similar anti-thrombotic effects of collagen-receptor inhibition, one would expect similar consequences of the inhibition of said receptors for cancer growth and metastasis. Administration of an antibody blocking alpha2beta1 integrin receptor supports extravasation of cancer cells, i.e. increases the number of cancer cells, which can leave the bloodstream and consequently this blocking increases the number of tumors formed (Hangan D. et al., Cancer Res. 1996, Vol. 1.56(13), 3142-3149). Similarly, the number of metastatic foci of B16 melanomas in lungs is higher in VWF-knockout mice as compared to wild-type mice, an effect which can be reversed by substituting the mice with VWF (Terraube V. et al., J. Thromb. Haemost. 2006, Vol. 4(3), 519-526). Accordingly, it was expected that the inhibition of GPVI would also have cancer promoting effects. The data summarized here now surprisingly demonstrate the opposite: the inhibition of the GPVI not only dramatically reduces the growth of cancer cells but also reduces metastasis of the respective cancer cells.

By “inhibition of GPVI” in the sense of the invention the following is meant: any prevention of the normal function of GPVI for example by preventing the binding of agonists, or by preventing GPVI activation by ligands or by inactivation or reversible or irreversible depletion of GPVI on the surface of cells or cell fragments.

By “inhibitor of GPVI” in the sense of the invention the following is meant: any compound which if administered to a mammal results in the “inhibition of GPVI” as defined above.

Inhibition of GPVI can result from a large variety of mechanisms such as an acquired GPVI deficiency, resulting from anti-GPVI auto-antibodies, or a genetic, congenital deficiency, where GPVI is not expressed or is expressed in a dysfunctional form with defective intracellular signalling and activation of endogenous platelet metalloproteinases resulting in ectodomain shedding (Arthur J. F. et al., Br. J. Haematol. 2007, Vol. 139(3), 363-372).

The active principle of the GPVI inhibitor of the invention may be small chemical compounds, peptides, polypeptides or monoclonal or polyclonal antibodies.

By way of non-limiting example inhibitors of GPVI can be classified as:
Ligands to GPVI which when bound to GPVI result in the depletion of GPVI from the cell surface such as
   antibodies and fragments thereof such as scFv, Fab, Fv, dAb, Fd or diabodies binding to GPVI, for example
      JAQ1 (EP20010101406)
      F1232-10-2, F-1232-21-1, F-1232-7-1, F-1232-19-1, F-1232-37-2, F-1232-18, F-1232-17-1, F-1232-18-3, F-1232-14-2, F-1232-24-1, F-1201-20, F-1232-43-3, F-1201-18, F1199-6, F1232-37-2, YA-Abs-88, YA-Abs-03, F-1249-18-2, F-1245-7-1, F-1246-1-1, F-1249-5-1, F-1249-20-1, F-1249-24-1, F-1249-30-1, F-1245-5-1, F-1246-6-2, F-1249-3-2, F-1245-4-1, F-1249-22-1, F-1251-1-1, F-1257-3-1, F-1232-37-2, F1239-6-1 (WO/2006/118350)
      F1239-1-3, F1239-2-3, F1239-4-2, F1239-5-3, F1239-7-1, F1239-8-1, F1239-8-1, F1239-10-2, F1239-11-1, F1239-15-3, F1239-16-3, F1239-17-2, F1239-18-1, F1239-19-1, F1239-22-2, F1239-22-3, F1239-23-1, (WO/2007/116779)
      hGP 5C4 (PCT/EP2004/013779)
      10B12, 16E12, 1C3, 8A1, 4H9, 4D5 (WO/2003/054020)
      8M14.3, 3F8.1, 9E18.3, 3J24.2, 6E12.3, 1P10.2, 4L7.3, 7H4.6, 9012.2 (WO 2001/000810)
Ligands to GPVI resulting in proteolytic inactivation of GPVI such as
   Activation of metalloproteinases (Bergmeier W. et al., Thromb. Haemost. 2004. Vol. 91(5): 951-958.
Ligands which inhibit the function of GPVI but do not cause the depletion of GPVI from the cell surface by preventing
   ligand receptor recognition and/or
   interaction with the extracellular matrix and/or
   interaction with collagen for example by shielding GPVI binding sites of collagen, by GPVI or fragments thereof (WO/2001/16321, WO/2006/131512, WO/2003/008454, WO/2001/000810, WO/2000/068377, WO/2000/68377) and/or
   platelet cell interaction
Ligands decreasing GPVI function independent of the mechanisms as detailed above, for example by inhibition of signalling cascades triggered by activated GPVI
   for example by preventing phosphorylation of proteins involved in the intracellular signalling pathways such as Syk Kinase, c-Src, protein kinase c, phospholipase Cγ2, Fc receptor γ

The inhibitors of GPVI of the invention can be manufactured by conventional chemical synthesis or in the case of monoclonal or polyclonal antibodies or fragments of antibodies by recombinant methods well known in the art (see for example Benny K. C. Lo, “Antibody engineering, methods and protocols, Humana press, 2003). Polyclonal antibodies can also be raised in animals by immunizing with GPVI or fragments of GPVI and subsequently be purified.

It is preferred to purify the inhibitors of GPVI of the present invention to greater than 80% purity, more preferably greater than 95% purity, and particularly preferred is a pharmaceutically pure state that is greater than 99.9% pure with respect to contaminating molecules, for example if the inhibitor of GPVI is a peptide or polypeptide from contaminating macromolecules, particularly other proteins and nucleic acids, and free of infectious and pyrogenic agents. Preferably, an isolated or purified inhibitor of GPVI of the invention is substantially free of other polypeptides.

The present invention provides an inhibitor of GPVI as described herein for the prevention and or therapy of cancer and also the use of such an inhibitor of GPVI in the manufacture of a medicament for the prevention and/or therapy of cancer. Therefore, according to another aspect of the present invention, a pharmaceutical formulation is provided comprising this inhibitor, which is suitable for the treatment of cancer, preferably for skin cancer, more preferably for melanoma, and most preferably for malignant cutaneous melanoma.

The inhibitors described in this invention can be formulated into pharmaceutical preparations for therapeutic use. The purified proteins may be dissolved in conventional physiologically compatible aqueous buffer solutions to which there may be added, optionally, pharmaceutical excipients to provide pharmaceutical preparations.

Such pharmaceutical carriers and excipients as well as suitable pharmaceutical formulations are well known in the art (see for example “Pharmaceutical Formulation Development of Peptides and Proteins”, Frokjaer et al., Taylor & Francis (2000) or “Handbook of Pharmaceutical Excipients”, 3^{rd} edition, Kibbe et al., Pharmaceutical Press (2000)). In particular, the pharmaceutical composition comprising the polypeptide of the invention may be formulated in lyophilized or stable soluble form. The polypeptide may be lyophilized by a variety of procedures known in the art. Lyophilized formulations are reconstituted prior to use by the addition of one or more pharmaceutically acceptable diluents such as sterile water for injection or sterile physiological saline solution.

Formulations of the composition are delivered to the individual by any pharmaceutically suitable means of administration. Various delivery systems are known and can be used to administer the composition by any convenient route. Preferentially the compositions of the invention are administered systemically. For systemic use, the therapeutic proteins of the invention are formulated for parenteral (e.g. intravenous, subcutaneous, intramuscular, intraperitoneal, intracerebral, intrapulmonar, intranasal or transdermal) or enteral (e.g., oral, vaginal or rectal) delivery according to conventional methods. The most preferential route of administration is intravenous administration for inhibitors of GPVI based on polypeptides. The formulations can be administered continuously by infusion or by bolus injection. Some formulations encompass slow release systems.

For inhibitors of GPVI based on small molecules oral administration is the most preferred mode of administration. Tablets and capsules for oral administration may contain conventional excipients such as binding agents, fillers, lubricants and wetting agents, etc. Oral liquid preparations may be in the form of aqueous or oily suspensions, solutions, emulsions, syrups, elixirs or the like, or may be presented as a dry product for reconstitution with water or other suitable vehicle for use. Such liquid preparations may contain conventional additives, such as suspending agents, emulsifying agents, non-aqueous vehicles and preservatives.

Formulations suitable for topical application may be in the form of aqueous or oily suspensions, solutions, emulsions, gels or, preferably, emulsion ointments. Formulations useful for spray application may be in the form of a sprayable liquid or a dry powder.

The inhibitors of GPVI of the present invention are administered to patients in a therapeutically effective dose, meaning a dose that is sufficient to produce the desired effects, preventing or lessening the severity or spread of the condition or indication being treated without reaching a dose which produces intolerable adverse side effects. The exact dose depends on many factors as e.g. the indication, formulation, and mode of administration and has to be determined in preclinical and clinical trials for each respective indication.

The pharmaceutical composition of the invention may be administered alone or in conjunction with other therapeutic agents. These agents may be incorporated as part of the same pharmaceutical.

Another aspect of the invention is the use of a composition comprising one or more isolated inhibitors of GPVI for the manufacture of pharmaceutical preparations for simultaneous, separate or sequential use in the therapy of cancer, preferentially in the therapy of skin cancer, even more preferentially in the treatment of melanoma.

Still another aspect of the invention is the use of inhibitors identifiable by the method described in Example 3 to inhibit also components of the signalling cascade downstream of GPVI.

Another embodiment of the invention is a method to identify inhibitors of GPVI comprising the steps of:

a) adding an appropriate amount of a potential inhibitor of GPVI to platelet rich plasma of a mammal, preferably obtained from a human or a standard laboratory animal species such as mice
b) optionally incubating the platelet rich plasma of step (a) with the potential inhibitor of GPVI
c) starting the aggregation of platelets by adding collagen related peptide or convulxin or less specific collagen to induce platelet aggregation by GPVI-mediated signalling
d) comparing the aggregation as determined in step (c) with the aggregation which is obtained when collagen related peptide or convulxin or less specific collagen is added to platelet rich plasma without adding the potential inhibitor of step (a).

If the aggregation of platelets is impaired when adding the potential inhibitor of GPVI as described in step (a) this compound is an inhibitor of GPVI according to the invention. The specificity of this inhibitor of GPVI can be further tested in control experiments where the GPVI inhibitor is applied to platelet rich plasma, preferably from the same organism as in the first step, but in which GPVI is deficient. Such a deficiency can be achieved by a genetic deficient of GPVI, such as GPVI knock-out mice (Kato K, Kanaji T, Russell S, et al., Blood. 2003; 102: 1701-1707) or by other approaches leading to a substantially decreased GPVI activity, for example silencing the gene or techniques such as siRNA. Such a deficiency would not necessarily need to be complete. If the compound added still impairs aggregation of such a preparation, when aggregation is induced by alternative agonists than collagen related peptide, convulxin or less specific collagen, by activating platelets via other receptors than GPVI, such as ADP, thrombin or arachidonic acid or similar, the compound is (though it inhibits GPVI) not specific for GPVI.

Yet another aspect of the invention is the use of a composition comprising one or more isolated inhibitors of GPVI for the manufacture of a combined pharmaceutical preparation for simultaneous, separate or sequential use in the therapy of cancer, preferentially in the therapy of skin cancer, even more preferentially in the treatment of melanoma.

A further aspect of the invention is a composition comprising one or more isolated inhibitors of GPVI and at least one further therapeutic compound which is not an inhibitor of GPVI for simultaneous, separate or sequential use in the therapy of cancer, preferentially in the therapy of skin cancer, even more preferentially in the treatment of melanoma.

Yet a further aspect of the invention is a combined composition comprising one or more isolated inhibitors of GPVI and at least one further therapeutic compound which is not an inhibitor of GPVI for simultaneous, separate or sequential use in the therapy of cancer, preferentially in the therapy of skin cancer, even more preferentially in the treatment of melanoma.

As GPVI inhibition has a proven antithrombotic effect and has been shown not to cause a bleeding risk, therapeutic interventions with GPVI inhibitors might be of special benefit for patients who must not be exposed to an increased risk of bleeding complications such as cancer patients undergoing conventional surgical interventions.

Especially for cancer patients GPVI inhibition may have synergistic effects from both the antithrombotic as well as the anti-cancer effect. Thus even more so as cancer patients suffer from an increased risk of thrombosis.

### FIGURES

FIG. 1: Reduction of cancer incidence in mice by GPVI inhibition as compared to negative (vehicle treated) control mice about two weeks after 5×10⁴ B16 cells i.v.

FIG. 2: Lungs of vehicle treated mice 2 weeks after 1×10⁶ B16 cells i.v.

FIG. 3: Lungs of mice with GPVI inhibition 2 weeks after 1×10⁶ B16 cells i.v.

FIG. 4: Number of lung colonies two weeks after 1.5×10⁶ B16 cells i.v. (n=5-10/group, individual results, group median connected by line)

FIG. 5: Survival rate of B16 infused mice, following treatment with vehicle (negative control) or following a limited period of GPVI inhibition, starting pre- or post-B16 injection (n=9-10/group)

FIG. 6: Bodyweight of B16 infused mice over time, following treatment with vehicle (negative control) or following a limited period of GPVI inhibition, staring pre- or post-B16 injection (mean, n=9-10/group)

### EXAMPLES

### Example 1

The goal of the first set of experiments was to assess whether the treatment of C57Bl6 mice with the monoclonal antibody JAQ1 which binds to GPVI could prevent the growth of B16 melanoma colonies in the lungs, following intravenous injection of B16 cells at three different cell numbers.

The model used to test this hypothesis was performed in the following manner. To propagate the cancerous cells, B16 cells were prepared under sterile conditions from a deep frozen stock, purchased from the American Type Culture Collection (ATCC). Cells were slowly thawed and re-suspended with culture medium. 1×10⁶ B16 cells checked for viability with tryptan blue. For a first propagation, they were injected subcutaneously into a C57Bl6 mouse. 2 weeks later the resulting subcutaneous tumor was removed and the tumor cells were separated by collagenase. Further propagation of the cells was performed in vitro. In brief, the isolated cells were transferred into culture dishes filled with respective medium. Once the cells had multiplied to a stage which was visibly detectable, the adherent cells were mobilized using trypsin. At this stage either another in vitro propagation cycle followed or the cells were injected i.v. into the tail vein of the C57Bl6 test mice to test the therapeutic effects of GPVI inhibitors. The mice (Charles River) were kept under standard housing conditions. The key endpoints of this animal model of skin cancer are the following:

1) percentage of animals in a treatment group found with tumors (incidence)
2) mortality (number of animals which died from the tumors or found moribund and were sacrificed)
3) the mean number of tumors and metastasis found on the lungs per animal in the respective treatment group.

While tumors also grow in the skin, they can best be quantified on the lungs of the mice, due to the circumstance that the contrast between the dark brown to black colour of the tumors represents a strong contrast to the light coloured lung tissue. Against the background of the dark skin such an analysis would be less reliably. In addition, the majority of the injected cells get trapped in the pulmonary microvasculature, adhering to the vascular endothelium.

The treatment schedule of the test mice was as detailed in Table 1. The dose of JAQ1 or saline (as negative control) vehicle was identical for all mice. The treatment started either 4 or 1 days prior to injection of the B16 cells and was continued as detailed in Table 1. Mice were injected with three different numbers of B16 cells, either 5×10⁴, 1×10⁶ or 1.5×10⁶. About two weeks later the animals were sacrificed and the number of B16 colonies in the lung were determined. A single treatment with JAQ1 depletes GPVI for a prolonged period of about 5 days (Nieswandt B. et al., J. Exp. Med. 2001, Vol. 193(4): 459-469). The repeated short interval schedule chosen insured that GPVI depletion was complete during the course of the experiments.

TABLE 1
Treatment groups

| | | | Number | |
|---|---|---|---|---|
| | | Dose/volume/ | of | |
| Set | Treatment | schedule/route | B16 cells | N (m/f) |
| A | saline | na/0.2 mL/20 g b.w./t = | 5 × 10⁴ | 11 (11/0) |
| | | d −1, d1, 3, 6 & 8/i.v. | | |
| | JAQ1 | 0.6 mg/kg/0.2 mL/20 g | 5 × 10⁴ | 11 (11/0) |
| | | b.w./t = d −1, d1, | | |
| | | 3, 6 & 8/i.v. | | |
| B | saline | Na/0.2 mL/20 g b.w./t = | 1 × 10⁶ | 11 (11/0) |
| | | d-4, d-1, d1, 3, 6 & 8/i.v. | | |
| | JAQ1 | 0.6 mg/kg/0.2 mL/20 g | 1 × 10⁶ | 11 (11/0) |
| | | b.w./t = d-4, d-1, d1, | | |
| | | 3, 6 & 8/i.v. | | |
| C | saline | na/0.2 mL/20 g b.w./t = | 1.5 × 10⁶ | 5 (5/0) |
| | | d-4, d-1, d1, 3, 6 and 8/i.v. | | |
| | JAQ1 | 0.6 mg/kg/0.2 mL/20 g | 1.5 × 10⁶ | 10 (0/10) |
| | | b.w./t = d-4, d-1, d1, | | |
| | | 3, 6 & 8/i.v. | | |

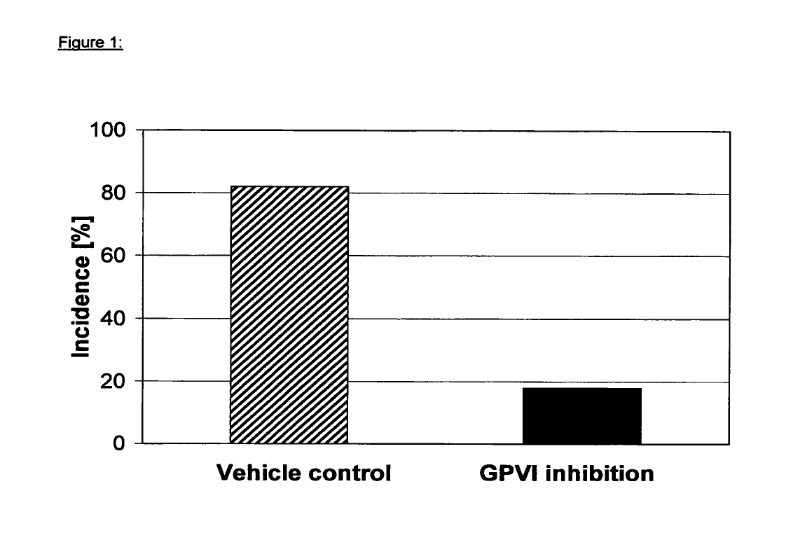

If the number of B16 cells injected was low (5×10⁴ B16 cells/mouse, set A), treatment with JAQ1, starting one day before i.v. infusion of B16 cells, dramatically reduced the number of animals, which developed B16 lung colonies from 82% to just 18% (Tab. 2, FIG. 1). The number of colonies in the lungs was similarly reduced by the treatment, as well.

TABLE 2
Lung colonies 14 days after 5 × 10⁴ B16 cells/mouse i.v.
(set A, n = 11/group, mean ± SD)

| | Incidence | Number of lung |
|---|---|---|
| Treatment | [%] | colonies |
| saline, t = d-1, d1, d3, d6 an d8 i.v. | 82% | 2.5 ± 1.9 |
| JAQ1, 0.6 mg/kg | 18% | 0.5 ± 1.2 |
| t = d-1, d1, d3, d6 an d8 i.v. | | |

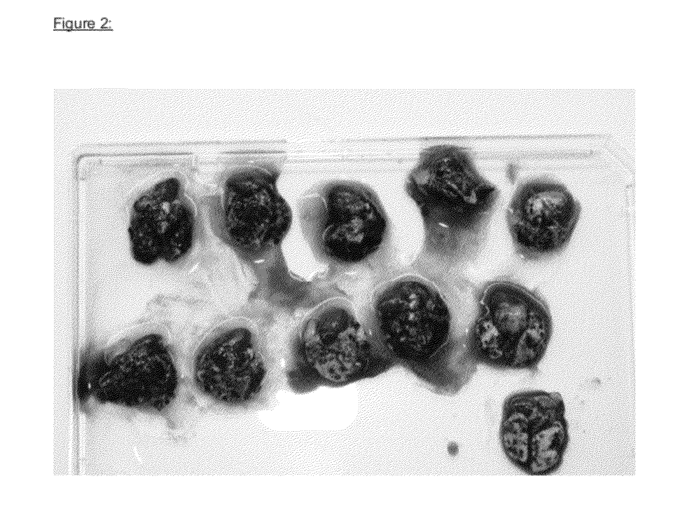

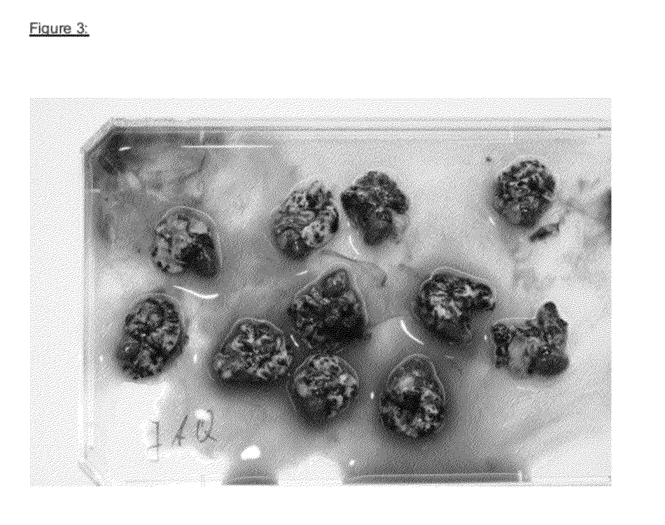

Upon increase of the number of injected B16 cells by a factor of 20× (1×10⁶ B16 cells/mouse, set B), all saline treated mice showed colonies, but their number was so high, that they could not be counted reliably. An additional difference to set A was that treatment started 3 days earlier than in set A, i.e. 4 days before B16 cell infusion. Also in this more aggressive situation with regard to the B16 infusion, JAQ1 treatment resulted in a clear reduction of B16 lung colonies. The number of colonies on the lungs of saline treated mice was so high (>100) that they could not be counted reliably, thus FIGS. 2 and 3 show pictures of the organs of vehicle and JAQ1 treated mice (Tab. 3) clearly showing the therapeutic effect of JAQ1 injection.

TABLE 3
Lung colonies two weeks after 1 × 10⁶ B16 cells/mouse i.v.
(set B, n = 11/group, mean ± SD)

| | Incidence | Number of |
|---|---|---|
| Treatment | [%] | lung colonies |
| saline, t = d-4, d-1, d1, d3, d6 an | 100% | >100 (uncountable) |
| d8 i.v. | | |
| JAQ1 IgG2a, 0.6 mg/kg | 100% | 92 ± 18 |
| t = d-4, d-1, d1, d3, d6 an d8 i.v. | | |

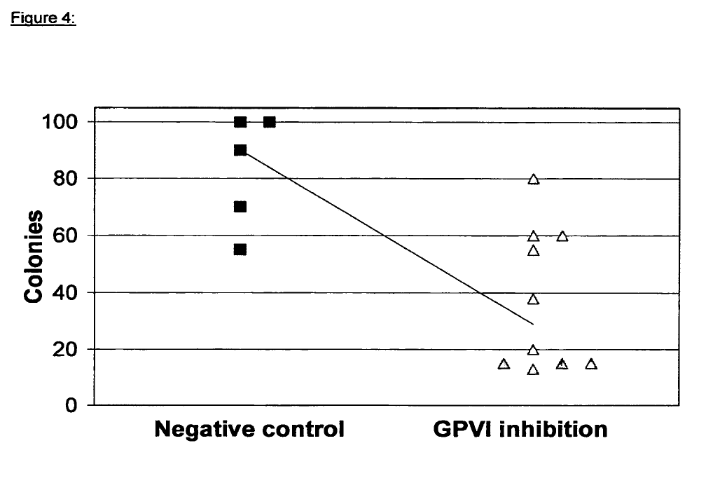

In a repeat experiment, to confirm the robustness of this finding against the background of incidental variations of the model, the B16 cells propagated turned out less malignant in vitro than for set B. This allowed a further rise to 1.5×10⁶ cells/mouse (set C). The treatment schedule was the same as with set B. Also at this higher cell load with less aggressive cells, JAQ1 treatment, starting 4 days before B16 cell infusion, resulted still in a clear reduction of lung colonies (Tab. 4, FIG. 4).

TABLE 4
Lung colonies two weeks after 1.5 × 10⁶ B16 cells/mouse i.v.
(set C, n = 5-10/group, mean ± SD)

| | Incidence | Number of lung |
|---|---|---|
| treatment | [%] | colonies |
| saline, t = d-4, d-1, d1, d3, d6 and | 100% | 83 ± 20 |
| d8, i.v. | | |
| JAQ1 IgG2a, t = d-4, d-1, d1, d3, d6 | 100% | 37 ± 25 |
| and d8, i.v. | | |

Taken together this set of data demonstrates that JAQ1 treatment reduces the number of melanoma colonies.

One may speculate by which mechanism, GPVI inhibition causes this protection. It may be mediated by several mechanisms. It may result from preventing the arrest of malignant cells injected at the start of the experiment. Reduction of tumors may however also be caused by preventing growth of arrested cells, which were released from initial tumors, which were generated by the transfusion at the start of the experiment, i.e. metastases. There is however no reason to assume that the origin of circulating cells defines their further fate, i.e. the initial transfusion already reflects the situation of metastasis by tumors growing in the mice. In addition it seems also plausible that the GPVI deficiency inhibits the growth of tumors, thus delays the timepoint when metastasis starts or that the process of releasing metastatic cells into circulation is inhibited by the GPVI deficiency.

### Example 2

The data summarized in the first example demonstrate that GPVI depletion not only dramatically reduces the growth of tumors but also minimizes metastasis. The receptor depletion was, however, induced prior to the injection of the malignant cells. Prophylactic GPVI inhibition with no evidence of cancer may represent, however, a rather rare situation for the potential clinical setting. The hypothesis tested in the second example was therefore whether GPVI inhibition is also efficient if started after transfusion of malignant cells thus not only allowing a prophylactic protection against cancer but also the treatment after of an already established cancer. To assess this question the clinically most relevant endpoint, i.e. mortality was used to determine efficacy, instead of determining the number of colonies at one specific point in time only. Moreover a worst case scenario of a short period of therapy was chosen. The C57Bl6 mice in this example were treated as detailed in Table 5. As a variation of example 1 and for a direct comparison of the potential impact of the starting point in time for therapy, the effect of JAQ1 was assessed by two treatment schedules. One started 4 days prior to transfusion of the malignant cells, as with example 1, the second schedule started 8 hours after transfusion of B16 cells, a point in time when no longer any freely circulating cancer cells are present. Thus presumably most if not all malignant cells have arrested or adhered to the endothelium or already invaded the surrounding tissue following their extravasation through the vessel wall. This is important in order to determine whether the treatment effect depends on the diminished retention of tumor cells in the pulmonary microvasculature or is also mediated by independent mechanisms such as those concerning extravasation and successful metastasis.

Treatment with JAQ1 was limited to about one week. A treatment scheme was designed to ensure a pronounced GPVI inhibition following administration. Within the period of about 3-4 days, inhibition achieved by such a single treatment is complete, and it starts to decay once an increasing number of fully competent platelets is released from their progenitor cells and inhibited platelets get cleared, thus their ratio in circulation changes accordingly. After about one week following the last treatment, the level of GPVI inhibition is rather small and presumably insignificant in this setting. Accordingly, GPVI inhibition was not maintained until mortality in this example but restricted to at least the first week following the transfusion of malignant cells. Therefore the major part of the observation period until mortality (about 2-3 of the typically 3-4 weeks until mortality), the animals in both treatment groups were actually untreated.

TABLE 5
Treatment groups

| | | Number of | |
|---|---|---|---|
| Treatment | Dose/volume/schedule/route | B16 cells | N (m/f) |
| saline | na/0.2 mL/20 g b.w./t = d −4, −1, | 5 × 10⁵ | 10 (10/0) |
| | d1, d3, d6/i.v. | | |
| JAQ1 | 0.6 mg/kg/0.2 mL/20 g b.w./t = | 5 × 10⁵ | 10 (10/0) |
| | d −4/i.v. | | |
| | 0.4 mg/kg/0.2 mL/20 g b.w./t = −1, | | |
| | d1, d3, d6/i.v. | | |
| JAQ1 | 0.6 mg/kg/0.2 mL/20 g b.w./t = | 5 × 10⁵ | 10 (10/0) |
| | +8 hours, d3, d6, d9/i.v. | | |

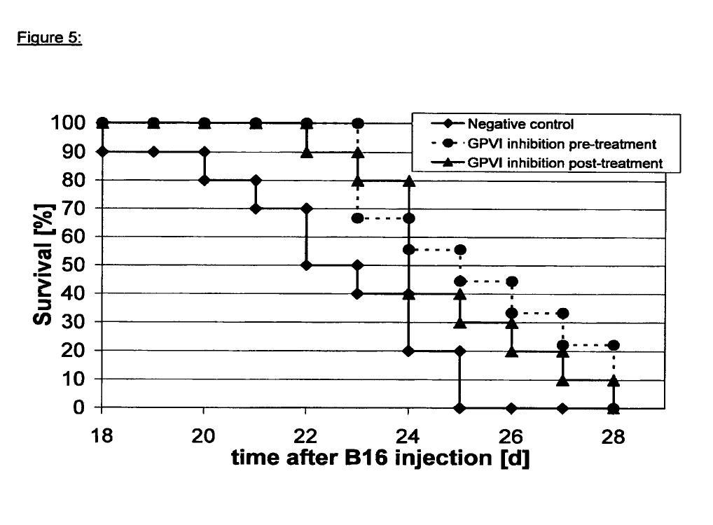

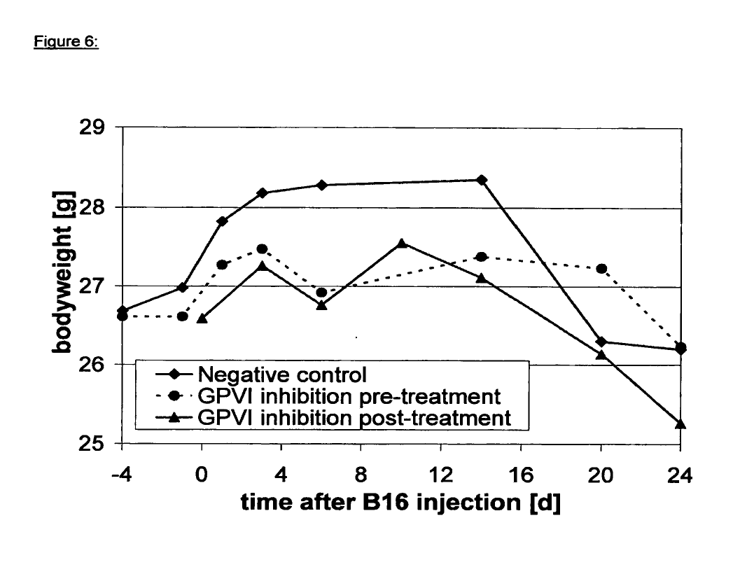

Although the period of therapy was quite short, GPVI inhibition clearly delayed the mortality (Tab. 6 and FIG. 5). Mortality rate appeared also quite similar, if GPVI inhibition started before or after infusion of B16 cells. The time course of bodyweight corresponded to the initial massive tumor growth, followed by a deteriorating general condition (FIG. 6).

TABLE 6
Survival rate after intravenous administration
of 5 × 10⁵ B16 melanoma cells

| Timepoint | | JAQ1 | JAQ1 |
|---|---|---|---|
| [day] | Vehicle | pre-treatment | post-treatment |
| d 17 | 100% | 100% | 100% |
| d 18 | 90% | 100% | 100% |
| d 19 | 90% | 100% | 100% |
| d 20 | 80% | 100% | 100% |
| d 21 | 70% | 100% | 100% |
| d 22 | 50% | 100% | 90% |
| d 23 | 40% | 67% | 80% |
| d 24 | 20% | 56% | 40% |
| d 25 | 0 | 44% | 30% |
| d 26 | 0 | 33% | 20% |
| d 27 | 0 | 22% | 10% |
| d 28 | 0 | 0 | 0 |

The pre-treatment effect in this example confirms the proof provided by the previous example 1, showing that inhibition of GPVI reduces tumor growth and/or metastasis.

Based only on Example 1 the anti-cancer effect of the inhibition of GPVI could also be explained by the anti-thrombotic effect of inhibiting GPVI, as cancer cell propagation might depend on the potential of cancer cells to induce coagulation which supports an increased retention of cancer cells in the microvasculature and thereby increases their chance to extravasate. For example a beneficial effect is reported for anticoagulated or fibrinogen-deficient animals (Amirkhosravi A. et al., J. Thrombosis Haemostasis, 2003, Vol. 1, 1972-1976; Palumbo J. S., Cancer Research, 2002, Vol. 62, 6966-6972).

In contrast, Example 2 shows, that there was no substantial difference whether GPVI inhibition was present at the time of malignant cell infusion or whether it was induced thereafter. Therefore, these data show that a maximal anti-cancer, anti-metastatic effect may be achieved even if therapy is initiated at a point in time when trapping of cancer cells in the lung and possibly even extravasation is already completed, e.g. at a point in time when the anti-thrombotic effect of a GPVI inhibitor might be less important.

This example also demonstrates that reduction of tumors is paralleled by a delayed mortality of GPVI depleted mice. Importantly, also in this worst case scenario of a quite short period of GPVI inhibition, treatment resulted in a clearly delayed mortality. With a continuous inhibition of GPVI the therapeutic efficacy is expected to be even more pronounced.

### Example 3

The third example illustrates that the therapeutic effects of GPVI inhibition are independent of the nature of the inhibitor. Inhibiting compounds can be identified by adding an appropriate concentration of a potential inhibitor to platelet rich plasma of a mammal, preferably obtained from a human or a standard laboratory animal species such as mice. Following a suitable incubation period, the aggregation of platelets is started by adding collagen related peptide or convulxin or less specific collagen, as described in EP1228768. These compounds are used, as they induce platelet aggregation by GPVI-mediated signalling (Nieswandt B, Watson S P., Blood. 2003; 15:102: 449-461). If aggregation is impaired, the compound or method used inhibits GPVI or a component of the downstream signalling cascade activated by GPVI. In a second step as a control experiment the potential GPVI inhibitor is applied to platelet rich plasma, preferably from the same organism as in the first step, but in which GPVI is deficient. Such a deficiency can be achieved by a genetic deficient of GPVI, such as GPVI knock-out mice (Kato K, Kanaji T, Russell. S, et al., Blood. 2003; 102: 1701-1707) or by other approaches leading to a substantially decreased GPVI activity, for example silencing the gene or techniques such as siRNA. Such a deficiency would not necessarily need to be complete. If the compound added still impairs aggregation of such a preparation, when aggregation is induced by alternative agonists, activating platelets via other receptors than GPVI, such as ADP, thrombin or arachidonic acid or similar, the compound is (though it inhibits GPVI) not specific for GPVI.

A specific example for a small chemical compound which inhibits GPVI is EXP3179, a metabolite of the angiotensin II type 1 receptor antagonist Losartan (Grothusen C, Umbreen S, et al, Arterioscler Thromb Vasc Biol. 2007 May; 27(5):1184-90). By repeated dosing or other approaches like continuous infusion, a plasma level in mice can be reached, ensuring an uninterrupted, high level of GPVI inhibition. Whether a high level of inhibition is achieved can be tested by quantifying collagen induced platelet aggregation.

In order to assess whether a GPVI inhibitor such as EXP3179 is efficient in preventing cancer such as melanomas as well, a group of about 10 mice are pre-treated EXP3179 until sufficient GPVI inhibition is reached. Preferably this is at least 25% or at least 50% or at least 75 or at least 90%. Suitable housing conditions and mouse strain for this study are detailed in example 1. Next B16 cells are intravenously transfused as detailed in example 1, possibly also a range of different cell numbers or cells with varying malignancy, as well. Under continued treatment with a GPVI inhibitor such as EXP3179 the mice are observed for an appropriate period, about one week or longer, while recording the survivals of the mice. At the end of the observation period the mice are sacrificed and the endpoints listed in example one are quantified. As reference a similarly large group of mice are kept under the same conditions and treated identically except that the transfusion solution does not contain B16 cells. The comparison with this control group will demonstrate that GPVI inhibition with an inhibitor such as EXP3179 is similarly efficient in reducing tumors and metastasis growth accompanied with improved survival, as with using an inhibitor such as JAQ1.

In a second approach, representing the clinically more relevant situation of treating a patient with a GPVI inhibitor such as EXP3179, after the diagnosis of cancer has been made can be studied. This is complementary to the prophylactic setting described in example one—with an inhibition being fully present at the time point when the cancer cells get transfused. In this truly therapeutic setting, summarized in example two, the induction of cancer (by transfusion of B16 cells) occurs prior to the treatment with a suitable GPVI inhibitor. Quantification of the endpoints remains the same. Also in this therapeutic situation a reduced tumor and metastasis growth is seen, accompanied by an improved survival rate.

A potentially alternative approach of assessing the benefit of GPVI inhibition with regard to methods for the prevention and treatment of cancer by inhibition of GPVI might be the use of GPVI knock-out mice. However there are several fundamental aspects making this approache different from a treatment of mice with intact GPVI leading to an inhibited GPVI function. Strictly, the use of a GPVI knock-out mice only reflects a patient with a disruption of the GPVI gene which is a rare situation (for review see: Arthur J F, Dunkley S, Andrews R K. Br J. Haematol. 2007 November; 139(3):363-72). The characteristic feature of such a case is that the GPVI deficiency is present already during development of the cancer. Thereby it reflects the situation of a patient under prophylactic GPVI inhibition treatment, but nevertheless developing a cancer. This becomes even more obvious in the case of a patient with complete GPVI deficiency (reflected by a GPVI knock-out mouse too). A therapy aiming to reduce GPVI activity in such a patient would be meaningless. In contrast the current invention shows that the inhibition of GPVI in individuals with GPVI activity which is initially not inhibited might have the potential not only to prophylactically prevent cancer but rather to treat cancer therapeutically after the diagnosis of cancer. As shown in example 2 the inhibition of GPVI is also efficient if that treatment is initiated following diagnosis of the disease, thus as therapeutic approach.

Taken together, it is therefore concluded that GPVI inhibition may not only be efficient as prophylactic treatment, but also in patients with established melanoma and in a stage, where metastasis is a prominent process. Since platelets are major players in the initiation of vascular remodelling (Massberg et al., J. Exp. Med., 2006, Vol. 203(5): 1221-1233) GPVI depletion may operate through interfering with or inhibiting several mechanisms such those involved in neoangiogenesis, required for cancer growth, or metastasis, i.e. dissemination, arrest or invasion of cancer cells. Accordingly, GPVI-depleting agents or GPVI-inhibiting agents or agents preventing the function of GPVI by other means can be used as novel anti-cancer/anti-metastatic agents. They might be of special benefit for patients who must not be exposed to an increased risk of bleeding complications.

1-26. (canceled)

27. A method to treat skin cancer comprising administering to a mammal in need thereof an effective amount of the pharmaceutical composition comprising an antibody against GPVI and a pharmaceutically acceptable carrier or excipient, wherein the antibody against GPVI is effective for treating skin cancer.

28. A method according to claim 27, where in the mammal is a human.

29. A method according to claim 28, where in the skin cancer is melanoma.

30. A method according to claim 28, wherein the skin cancer is malignant cutaneous melanoma.

31. A method to treat skin cancer comprising administering to a mammal an effective amount of the pharmaceutical composition comprising an antibody against GPVI, at least one additional therapeutic compound, and a pharmaceutically acceptable carrier or excipient, wherein the additional therapeutic compound is not an antibody against GPVI, and wherein both the antibody against GPVI and the additional therapeutic compound are effective for treating skin cancer.

32. A method according to claim 27, further comprising administering to the mammal an effective amount of at least one additional therapeutic compound, wherein the additional therapeutic compound is not an antibody against GPVI, and wherein the additional therapeutic compound is effective for treating skin cancer.

33. A method according to claim 32, wherein the antibody against GPVI and the additional therapeutic compound are administered simultaneously, separately, or sequentially.

34. A method to identify a potential GPVI inhibitor, comprising the steps of:
a). inducing aggregation of platelets in platelet rich plasma of a mammal in the absence of the potential GPVI inhibitor;
b). inducing aggregation of platelets in platelet rich plasma of the mammal in the presence of the potential GPVI inhibitor;
c). determining the aggregation of platelets in steps a) and b);
d). comparing the aggregation as determined in step c), wherein a decreased aggregation in step b) as compared to the aggregation in step a) indicates that the potential GPVI inhibitor inhibits GPVI function.

35. A method according to claim 34, wherein the aggregation of platelets is induced by adding collagen related peptide, convulxin, or collagen to the platelet rich plasma in the absence or presence of the potential GPVI inhibitor.

36. A method according to claim 27, wherein the antibody is a monoclonal antibody.

37. A method according to claim 36, wherein the monoclonal antibody is JAQ1.

38. A method according to claim 31, where in the mammal is a human.

39. A method according to claim 38, where in the skin cancer is melanoma.

40. A method according to claim 38, wherein the skin cancer is malignant cutaneous melanoma.

41. A method according to claim 38, wherein the antibody is a monoclonal antibody.

42. A method according to claim 41, wherein the monoclonal antibody is JAQ1.

43. A method according to claim 32, where in the mammal is a human.

44. A method according to claim 43, where in the skin cancer is melanoma.

45. A method according to claim 43, wherein the skin cancer is malignant cutaneous melanoma.

46. A method according to claim 43, wherein the antibody is a monoclonal antibody.

47. A method according to claim 46, wherein the monoclonal antibody is JAQ1.
